(19) United States
(12) Patent Application Publication
(10) Pub. No.: US 2026/0137955 A1
(43) Pub. Date: May 21, 2026

(54) PHOTODISINFECTION DEVICE

(71) Applicant: ToeFX Inc., Hamilton (CA)

(72) Inventors: Showkat Monika Yazdanian, Hamilton (CA); Irit Itzhaki Van-Ham, Vaughan (CA)

(21) Appl. No.: 18/705,890

(22) PCT Filed: Apr. 5, 2023
(86) PCT No.: PCT/CA2023/050466

§ 371 Date: Apr. 29, 2024

Related U.S. Application Data
(60) Provisional application No. 63/327,405, filed on Apr. 5, 2022.
(60) Provisional application No. 63/393,110, filed on Jul. 28, 2022.

(51) Int. Cl. A61N 5/06; A61B 5/00

(52) U.S. Cl. CPC A61N 5/0624; A61B 5/0036; A61B 5/0071; A61B 5/0077; A61B 5/742; A61N 2005/0642; A61N 2005/0652

(57) ABSTRACT

Provided is a photodisinfection and visualization device for onychomycosis. The device includes a housing for substantially enclosing a foot, including the entirety of all toes, the housing having an opening for receiving the foot into a cavity of the housing. The device includes a light source array embedded in at least a top surface of the cavity for directing light onto toes of the foot, wherein the light source array emits at least one wavelength of light for activating a fluorophore or photosensitizer formulation applied to the toes. The device may include a levelling mechanism disposed within the cavity, for aligning the toes in a substantially horizontal plane, such that the toes are equidistant from the light source array ensuring that all toenails receive an equivalent dosage of light. The device may include a basin disposed within the cavity for containing an iontophoresis solution for combination with photodisinfection.

### TECHNICAL FIELD

The embodiments disclosed herein relate to phototherapy, and, in particular to a combined photodisinfection and visualization device for uniform illumination of a target area.

### INTRODUCTION

Phototherapy has been used to treat certain ailments. For example, inflammation can be reduced by shining blue light onto skin to illicit an anti-inflammatory response in tissues. Photodisinfection entails the administration of light to destroy or weaken microbes. Photosensitizer compounds in topical medications may be activated by shining a specific wavelength or wavelengths of light onto the treated area to produce reactive oxygen species that have anti-microbial, anti-fungal or other therapeutic effects. For example, it has been shown that anti-fungal photosensitizer compounds excitable by red light may be effective for treatment of onychomycosis.

Existing photodisinfection devices include a light source, for example, a bulb (lamp), a laser or LEDs. In the case of a lamp or a laser, the light source may produce considerable heat during operation, thus becoming uncomfortably hot or unsafe for a user to use for long durations. High intensity lasers can also cause damage to eyesight if the laser light is shined or reflected into the eye of the user.

Bulbs and LED light sources are often omnidirectional and do not provide uniform illumination of a target area, particularly if the target area is contoured, undulated, or non-planar. Thus, it is difficult to accurately measure the dosage of light that is received at the target area to assess the therapeutic effect. A further difficulty is some phototherapy devices are hand-operated, requiring the user to direct the light source onto the target area, which can also result in non-uniform illumination when the user moves their hand. Finally, identification or confirmation of onychomycosis is a well-known clinical problem, as onychomycosis can be similar in appearance to the presentation of other ailments such as nail trauma or psoriasis.

Accordingly, there is a need for a new photodisinfection and visualization device to provide uniform illumination of a target area and remains cool when operating for long durations.

### SUMMARY

According to an embodiment, there is a combined photodisinfection and visualization device for onychomycosis. The device includes a housing having at least one open end for receiving the foot into a cavity of the housing. The device includes a light source array embedded in at least a top surface of the cavity for directing light onto toes of the foot, wherein the light source array emits at least one wavelength of light for exciting a fluorophore absorbed into the toenails. The device includes a detachable levelling mechanism disposed within the cavity, for aligning the toes in a substantially horizontal plane, such that the toenails are equidistant from the light source array ensuring that all toenails receive an equivalent dosage of light.

The light source array comprises a plurality of light emitting diodes (LEDs). The plurality of LEDs preferably includes a first subset of LEDs emitting a first wavelength of light, and at least a second subset of LEDs emitting a second wavelength of light. Each LED is provided with a focusing lens to concentrate light emitted by the LED to a ˜45-degree beam angle. According to various embodiments, the light source array may be further embedded in opposing side walls of the cavity and/or a back wall of the cavity. According to an embodiment, the device includes extendable feet, whereby the distance between the light source array and the toenails may be varied by extending or retracting the feet.

The levelling mechanism may be adjustable in height to vary a distance between the light source array and the toenails. According to an embodiment, the levelling mechanism includes a ramped platform with an adjustable incline of at least 2 degrees. According to an embodiment, the levelling mechanism includes a hinged segment and spacers to lift and spread the toes to correct for irregularities in toe alignment and curvature.

The photodisinfection and visualization device further includes a cooling system having one or more heatsinks connected to the light source array. The cooling system may include one or more fans positioned to force air over the heatsinks. The cooling system may include a fan for blowing air into the cavity to cool a user's foot during operation of the device.

According to an embodiment, the device includes a detachable basin disposed within the cavity to submerge the user's foot in water or an iontophoresis solution.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is a perspective view of a photodisinfection and visualization device, according to one embodiment, shown in relation to a user's foot;

FIG. 2 is a front view of the device of FIG. 1;

FIG. 3 is a bottom view of the device of FIG. 1;

FIGS. 4A and 4B are perspective and side views, respectively, of a levelling mechanism, according to an embodiment;

FIGS. 4C and 4D are front and side views, respectively, of the levelling mechanism of FIG. 4B within the device of FIG. 2;

FIG. 4E is a perspective view of an adjustable levelling mechanism, according to another embodiment, shown in relation to a user's foot;

FIGS. 5A and 5B are perspective and side views, respectively, of a basin, according to an embodiment;

FIG. 5C is a diagram of iontophoresis components, according to an embodiment;

FIG. 6A is a perspective view of a cooling system in the device of FIG. 1, according to an embodiment;

FIGS. 6B-6D are top, side and rear views, respectively, of the cooling system of FIG. 6A; and

FIGS. 7A and 7B are perspective and side views, respectively, of a light source array, according to an embodiment.

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

A description of an embodiment with several components in communication with each other does not imply that all such components are required. On the contrary a variety of optional components are described to illustrate the wide variety of possible embodiments of the present invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

To assist in understanding the scope and content of the present disclosure, selected terms are defined directly below. Unless defined otherwise, all technical and scientific terms used herein have the same meaning as commonly understood by one of ordinary skill in the art to which the present disclosure pertains.

The term “photodisinfection” as used herein refers to a process in which microbes are irradiated with light sources at specific wavelengths. These wavelengths can create reactive oxygen species that destroy the microbes. Although photosensitizers are often used during photodisinfection, any mention of photodisinfection should be understood to reference only the device providing the light source(s).

The terms “LED” and “LEDs” as used herein refers to a light-emitting diode providing a semiconductor light source. In the present invention, it should be understood that “LED” could refer to miniature, standard, or high-power LEDs emitting wavelengths of light, including wavelengths in the visible light spectrum.

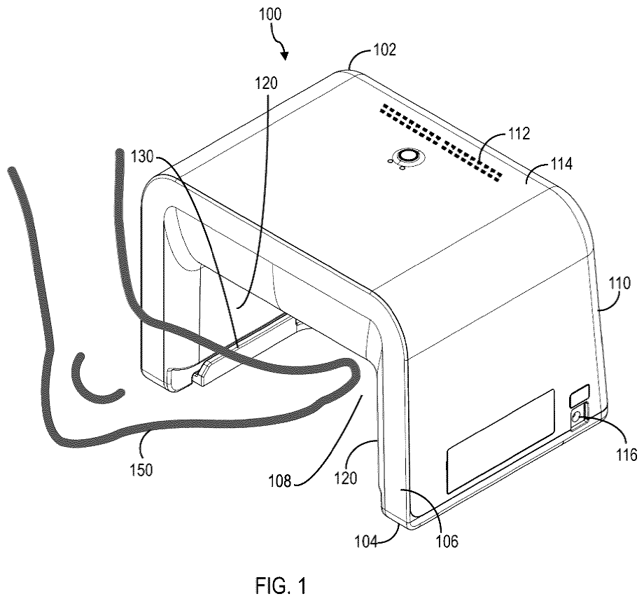

Referring to FIG. 1, shown therein is a photodisinfection and visualization device 100, according to an embodiment. The device 100 is shown in relation to a user's foot 150. The device 100 includes a housing 102 for containing a light source array and other components described below. The housing 102 is preferably constructed of a hard, durable, heat resistant plastic. The housing 102 is generally rectangular in shape and includes an open end 106, a cavity 108 and a closed end 110. According to other embodiments, the housing 102 may include a second open end rather than the closed end 110.

The cavity 108 is sized to accommodate at least one foot 150 of the user, such that the housing 102 substantially encloses at least the lateral, medial and dorsal forefoot regions of the foot 150 (including the entirety of the user's toes) when the user's foot 150 is inserted into the cavity 108 through the open end 106. When the user's foot is inserted into the cavity 108, the toes of the foot 150 are positioned in substantially the center of the cavity 108. According to other embodiments, the cavity 108 and the device 100 may be sized to enclose both feet of a user.

Prior to use, the device 100 is preferably placed on a floor to easily insert the user's foot 150 into the cavity 108. The device 100 includes an open base 104 allowing for the user's foot 150 to rest on the floor when inserted into the cavity 108. The device 100 includes vent openings 112 in a top cover 114 of the housing 102 for drawing air into the housing 102 by a cooling system described below. The device 100 includes a power port 116 for connecting a power cable from an AC power source (e.g., a wall outlet). According to various embodiments, the device 100 may further include a display port for connecting an external display and/or terminals for connecting iontophoresis electrodes (not shown).

According to an embodiment, the device 100 is for combined visualization and photodisinfection of onychomycosis. Onychomycosis is a fungal infection of the nail that degrades toenails causing discomfort and inflammation of the nailbed, toes and foot. In this exemplary application, the device 100 provides dual wavelength illumination of the toenails to visualize and photodisinfect onychomycosis.

Illumination of the foot/toenails with blue light is performed to reduce inflammation of the foot, toes and nailbed and for photodisinfection of onychomycosis in the nailbed. The anti-inflammatory and anti-fungal photodisinfection effects of blue light have been described previously.

It is important to first identify the location(s) and severity of onychomycosis infection for effective photodisinfection. Illumination of the toenails with red light, in combination with a red light-excitable fluorophore (e.g., Methylene Blue) applied to the toenails and absorbed into the toenail, may be performed to selectively stain/mark areas of onychomycosis infection, as described in U.S. Provisional Patent Application No. 63/393,110 to the same inventors, the entirety of which is incorporated herein by reference. The greater the severity infection, the more the fluorophore will localize to the infected area(s) and the higher the resulting fluorescence will be. In some cases, the fluorescence from the fluorophore may be detectable by eye.

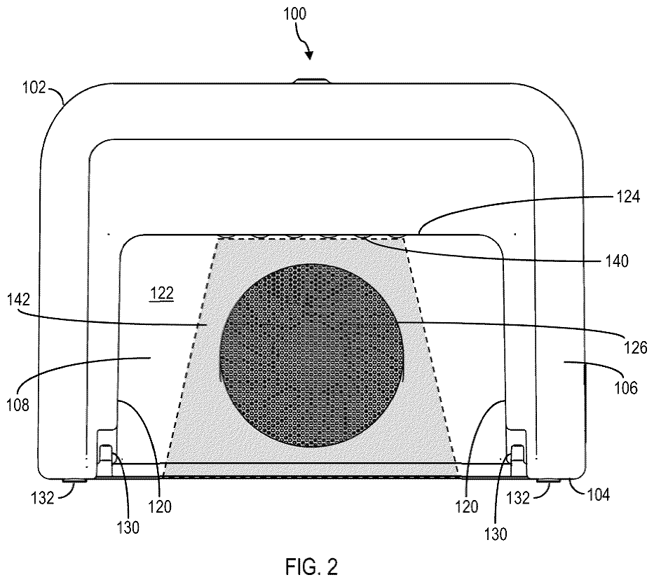

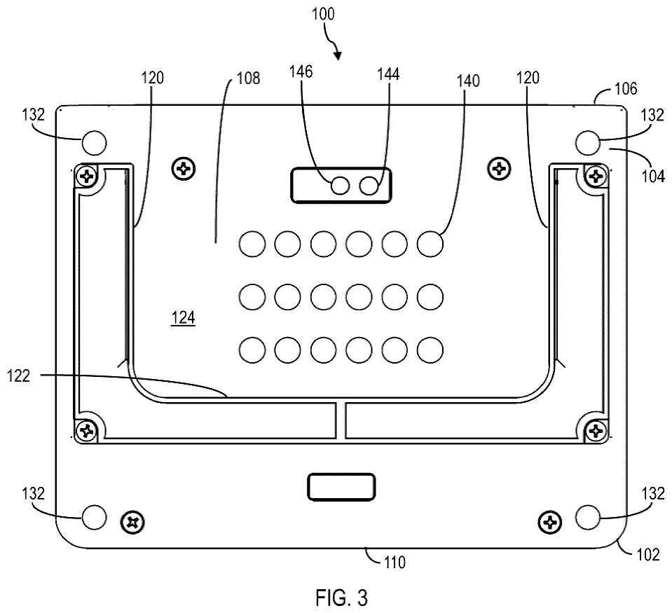

Referring to FIGS. 2 and 3, the device 100 includes a light source array 140. The light source array 140 provides the source(s) of light for the visualization and photodisinfection of onychomycosis. The light source array 140 includes a plurality of LEDs. The LEDs may be arranged in rows and columns, or other arrangements, with the LEDs being spaced at increments between 0.5 cm and 2 cm. LEDs are preferred over lasers or lamps for illumination, since LEDs are generally cheaper, have lower power requirements and do not generate as much heat during illumination compared to lasers or lamps.

The light source array 140 is preferably embedded in a top surface 124 of the cavity 108 and orientated to shine light downward toward the floor to form a region of uniform illumination 142 (shaded region in FIG. 2). The region of uniform illumination 142 is substantially in the center of the cavity 108 and covers a horizontal plane of at least 25 cm² at the narrowest part of the region of illumination 142.

According to other embodiments, the light source array 140 may be further embedded in opposable side walls 120 and/or a back wall 122 of the cavity 108, in addition to the top surface 124, to direct light to the region of uniform illumination 142 from the side walls 120 and/or the back wall 122 of the cavity 108. Advantageously, this provides for light to be directed to the region of uniform illumination 142 from multiple directions to ensure the entirety of the target area(s) (e.g., the toes/toenails on the user's foot), are illuminated, compared to unidirectional illumination wherein contours of the foot/toe/nail may prevent complete illumination of the target area(s).

The back wall 122 includes a vent 126 for air from the cooling system to blow into the cavity 108 to cool the user's foot during operation of the device 100. This may be particularly beneficial to prevent the user's foot from becoming uncomfortably hot when the device 100 is used for prolonged periods (i.e., longer than 5 minutes). The vent 126 is positioned in the center of the back wall 122 and aligned to blow air through the region of uniform illumination 142.

The device 100 includes extendable legs 132 in the open base 104 which can be extended or retracted, as required, to raise or lower the housing 102 relative to the floor, to vary a distance between the light source array 140 and the user's foot/toes. Depending on the wavelength(s) and intensity of light emitted by the light source array 140, there may be a minimum effective distance for sufficient illumination of the toes/toenails.

The side walls 120 of the cavity 108 and the open end 106 may include graduations or markings (not shown) to indicate the position of the region of uniform illumination 142 to aid in aligning the user's foot/toes with the region of illumination 142. The graduations or markings may further provide for estimating/measuring a distance between the toes/toenails and the light source array 140.

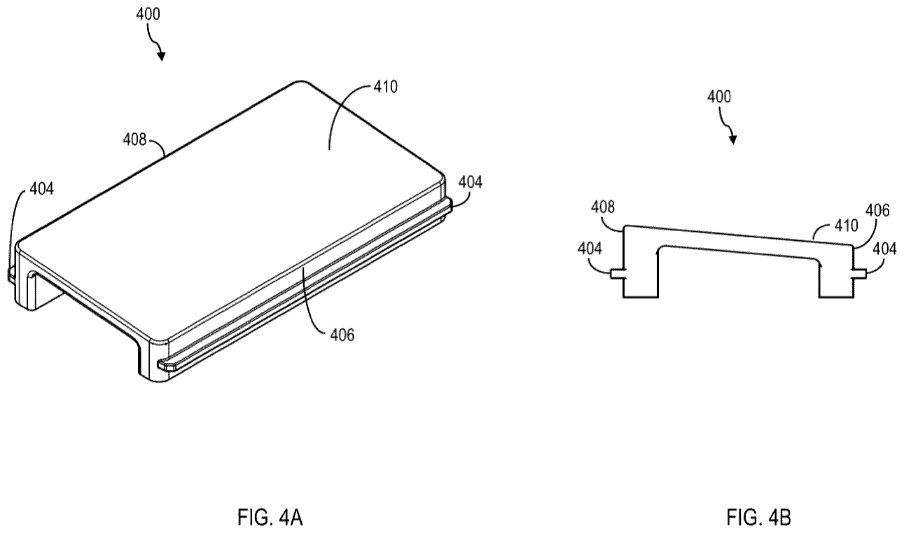

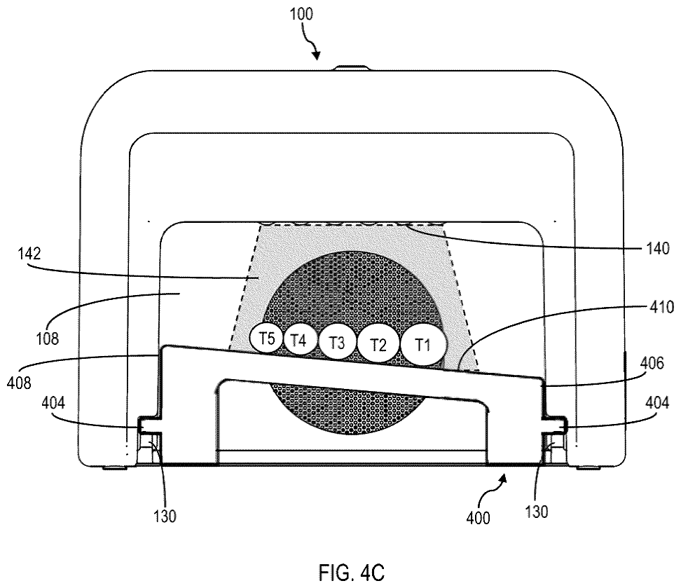

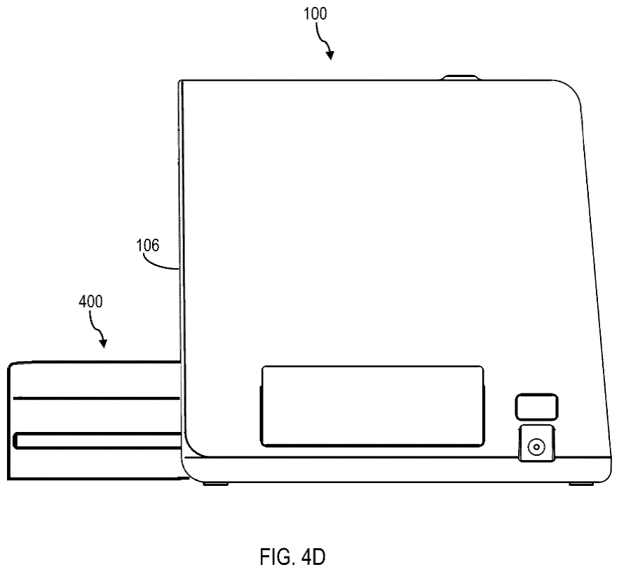

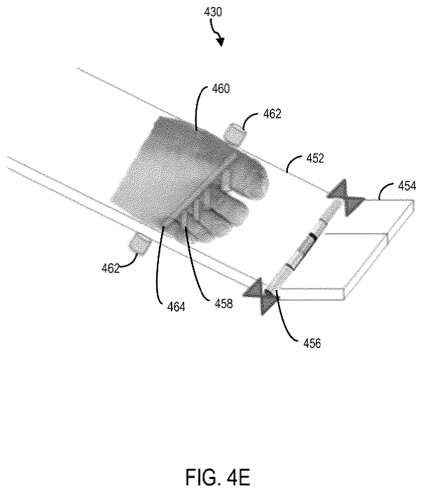

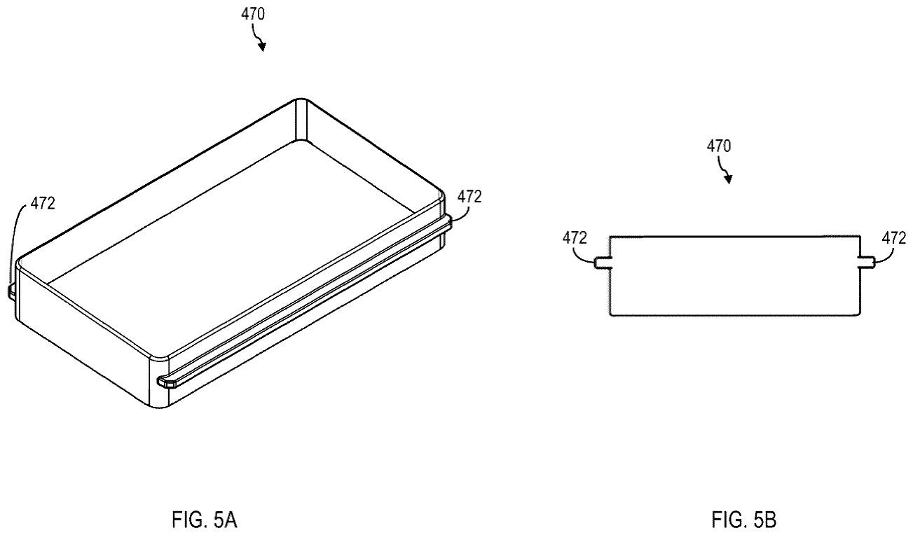

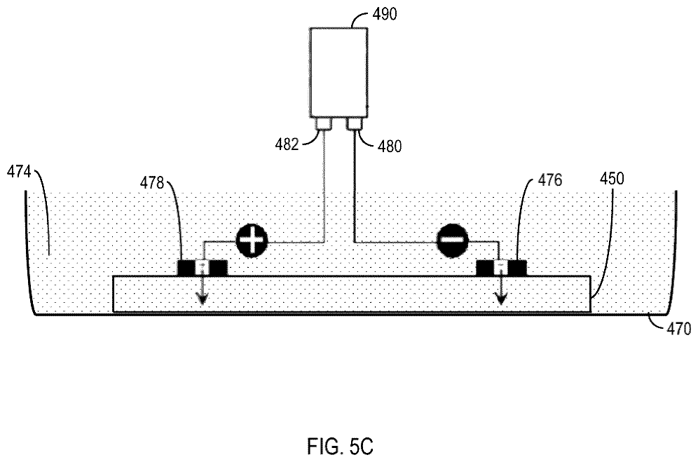

Referring to FIGS. 1 and 2, the device 100 includes opposable recessed rails 130 in the side walls 120 of the cavity 108 for mounting components such as a levelling mechanism (FIGS. 4A-4D) or a basin (FIGS. 5A-5C).

Now referring to FIG. 3, according to some embodiments, the device 100 may include a camera 146 disposed within the cavity 108 for viewing the foot/toes of the user when inserted into the cavity 108, to aid in alignment of the foot/toes with the region of uniform illumination 142, and to measure a distance between the toes/toenails and the light source array 140. The image from the camera 146 may be viewed on a connected external display (e.g., a monitor, a tablet, a smartphone, or the like). The device 100 may include display port(s) or Bluetooth connectivity for connecting to the external display.

The light source array 140 may be used to stimulate/excite a photosensitizer or a fluorophore in a topical formulation applied to the foot/toes. According to an embodiment, the device 100 includes a fluorescence sensor 144 disposed within the cavity 108, to detect fluorescence emitted by a fluorophore. The fluorescence sensor 144 may be a spectrophotometer configured to quantify the fluorescence emission by the fluorophore, e.g., Methylene Blue. A reading from the fluorescence sensor 144 may be displayed on a connected external display (e.g., a monitor, a tablet, a smartphone, or the like).

Referring to FIGS. 4A and 4B, shown therein are top and side views, respectively, of a levelling mechanism 400, according to an embodiment. The levelling mechanism 400 is used to compensate for foot/toe size and maintain the toenails of the user in a substantially horizontal plane within the cavity of the device (i.e., within the region of uniform illumination).

The levelling mechanism comprises a ramped platform 410, having an incline of at least 2 degrees between a lower end 406 and a higher end 408. According to some embodiments, the incline may be adjustable to up to 10 degrees to correct for the typical 1 to 10-degree discrepancy between the first toe (big toe) and fifth toe (pinky toe) that is typical in human feet. The levelling mechanism 400 includes tabs 404 for engaging the recessed rails 130 to attach the levelling mechanism 400 within the cavity 108 as shown in FIG. 4C. When attached, the levelling mechanism 400 may extend out of the cavity 108 through the open end 106 as shown in FIG. 4D. The levelling mechanism 400 is preferably constructed of a hard, durable, heat resistant plastic.

Referring to FIG. 4C, when the levelling mechanism 400 is used with the device 100, the foot of the user is placed on the ramped platform 410 to substantially align the toes of the foot in a horizontal plane within the region of uniform illumination 412 such that the toenails of all toes (shown as white circles, Tn), are substantially equidistant to the light source array 140 and each toenail receives an equivalent dosage of light. The levelling mechanism 400 may include graduations or markings to aid in aligning the toes/toenails with the region of uniform illumination 142. According to some embodiments, the levelling mechanism 400 may be adjustable in height to vary a distance between the light source array 140 and the user's toes/toenails.

As shown in FIG. 4C, the levelling mechanism 400 is oriented for placing the user's left foot thereon, with the fifth toe, T5, being placed higher on the ramped platform 410 closer to the higher end 408, and the first toe, T1, being placed lower on the ramped platform 410 closer to the lower end 406. For use with the right foot, the levelling mechanism is removed, turned 180 degrees and re-inserted into the cavity 108, so that the ramped platform 410 is inclined from left to right, for placing the user's right foot thereon, with the fifth toe, being placed higher on the ramped platform 410 closer to the higher end 408, and the first toe, being placed lower on the ramped platform 410 closer to the lower end 406, to align all the toenails in a substantially horizontal plane within the region of uniform illumination 142.

Referring to FIG. 4E, shown therein is a perspective view of an adjustable levelling mechanism 430, according to another embodiment, shown in relation to a user's foot 460. The adjustable levelling mechanism 430 may be employed for users afflicted by arthritis or other deformative ailments that cause irregularities in the alignment and curvature of the toes.

The levelling mechanism 430 includes a flat segment 452 and a hinged segment 454 connected by a spring-biased hinge 456. The hinged segment 454 is biased by the hinge 456 to be elevated relative to the flat segment 452 until depressed by the user's toes. When depressed by the toes, the spring-biased raised segment 454 exerts an upward force on the tips of the toes, to lift and straighten the toes so that all toes will be positioned in a substantially horizontal plane to receive an equivalent dosage of light.

The adjustable levelling mechanism 450 includes adjustable spacers 458 disposed on the flat segment 454 for spreading the toes. The position of the adjustable spacers 458 may be adjusted to vary the spacing of the toes. The adjustable spacers may be secured to the levelling mechanism 450 by a crossbar 464 and fasteners 462.

Referring to FIGS. 5A and 5B shown therein is a basin 470, according to an embodiment. The basin 470 may be provided as an optional, detachable accessory for the device 100. The basin 470 includes tabs 472 for engaging the recessed rails 130 of the device 100 to attach the basin 470 within the cavity 108 in the same manner as the levelling mechanism 400 shown in FIGS. 4C, 4D. When attached, the user's foot is placed in the basin 470.

The basin 470 is used for holding water or an iontophoresis solution containing a drug and/or a fluorophore. The user's foot is placed in the basin 470 at least partially immersing the user's foot in the water/solution therein. The water/solution may provide a cooling effect for the user's foot during prolonged use of the device 100. The basin 470 may further be used for iontophoresis in combination with photodisinfection as described below.

Referring to FIG. 5C, shown therein is a diagram of iontophoresis components, according to an embodiment. lontophoresis may assist the penetration of a topically-applied fluorophore, or a charged antimicrobial compound, into the skin or toenails of the foot 450. Iontophoretic chambers or skin electrodes acting as an anode 476 and a cathode 478 are placed on the user's foot or toenails 450. A solution 474 containing a charged antimicrobial compound or fluorophore is poured into the basin 470 and the basin 470 is inserted into the cavity of the device.

The user's foot 450 is placed in the basin 470, at least partially immersing the foot 450 in the solution 474. The anode 476 and the cathode 478 are connected to respective negative and positive terminals 480, 482 of a power supply 490 to commence iontophoresis. The power supply 490 may be an external power supply or power terminals on the device 100. During or after the iontophoresis, the light source array may be switched on to commence photodisinfection.

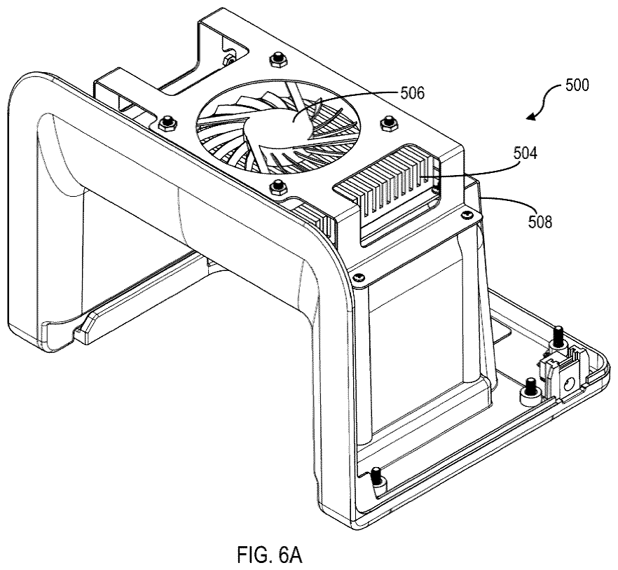

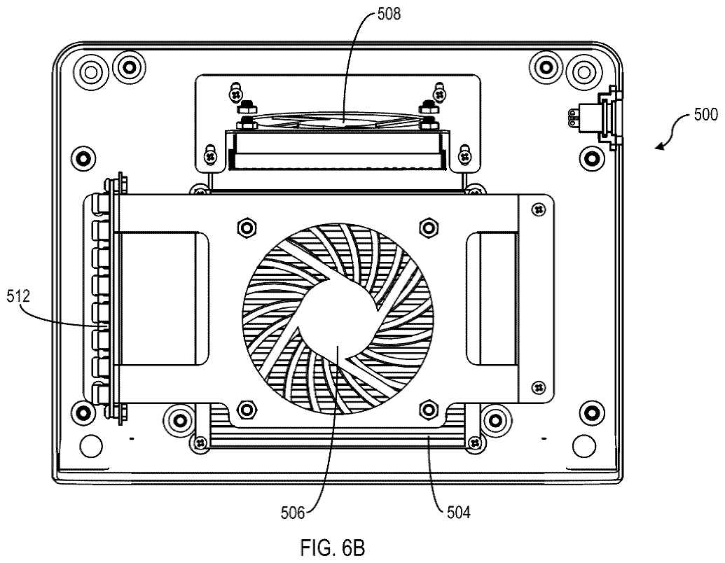

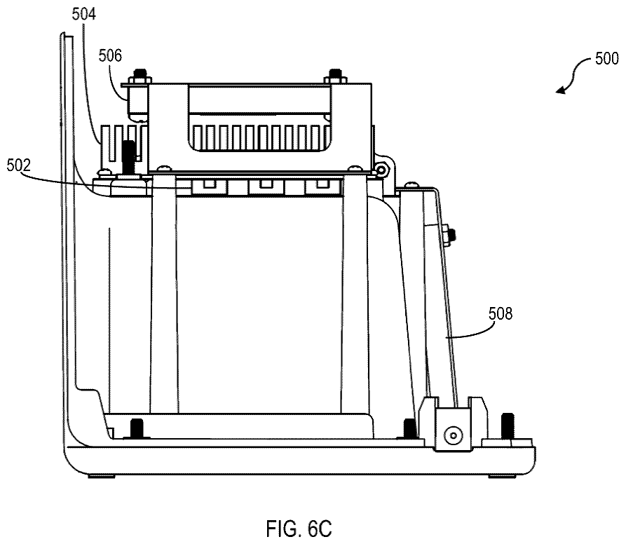

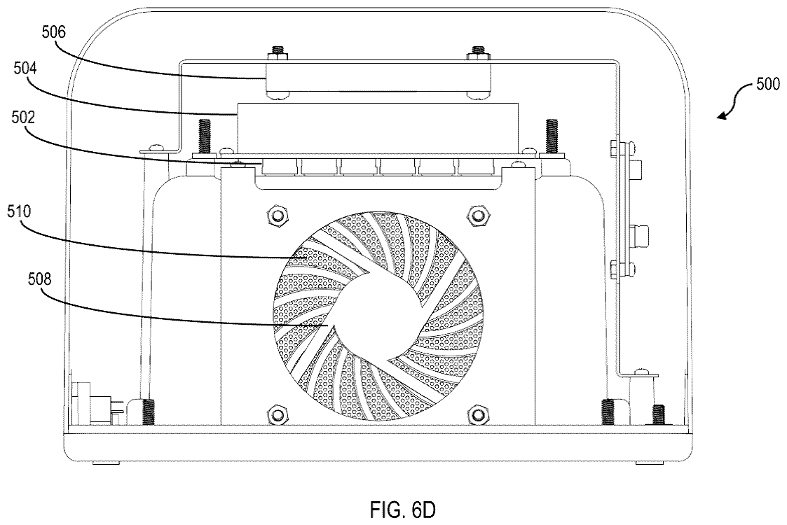

Referring to FIGS. 6A-6D, shown therein are perspective, top, side and rear views, respectively, of a cooling system 500 in the photodisinfection and visualization device 100 shown in FIG. 1, according to an embodiment. The cooling system 500 is positioned behind the light source array 502 within the housing of the device to cool/dissipate heat generated by the light source array 502 during operation. The cooling system 500 may also blow air into the cavity through the vent 510 (i.e., vent 126 in FIG. 1) to cool the user's foot.

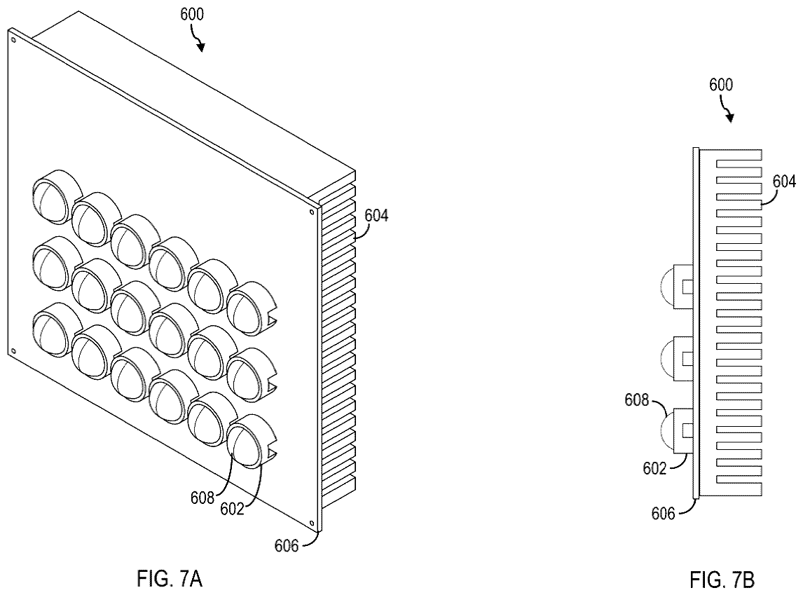

The cooling system 500 includes a heatsink 504 constructed of thermally conductive material such as graphite, aluminum, other plated or laminated metal, carbon fiber or metal-fiber composites. According to an embodiment, the heatsink 504 may be disposed as multiple rigid plates behind each row of LEDs in the light source array 502 (see FIGS. 7A and 7B). According to another embodiment, the heatsink 504 may be disposed as a lattice structure behind the light source array 502. A thermally conductive adhesive/substrate may be interposed between the heatsink 504 and the light source array 502 to aid in conducting heat from the light source array 502 to the heatsink 504.

The cooling system 500 includes at least one fan 506 positioned adjacent to the heatsink 504 to force air over the heatsink 504 to further aid in dissipating heat from the light source array 502. Generally, the heatsink and the fan 506 are configured to maintain an operating temperature of the light source array 502 between 30-60° C. to prevent the device from overheating. The fan 506 may be configured to operate continuously while the light source array 502 is on, or may be configured to start blowing when the temperature of the light source array 502 exceeds a threshold temperature.

The cooling system 500 further includes at least a second fan 508 positioned to blow air through the vent 510 into the cavity to cool the user's foot. The fan 508 may be particularly beneficial during prolonged use of the device (e.g., longer than 5 minutes of continuous use) to keep the user's foot cool and comfortable. The fan 508 may be configured to operate when the light source array 502 is on, or may be connected to a switch on the housing to allow the user to turn the fan 508 on when desired.

Referring to FIGS. 7A and 7B, shown therein is a light source array 600, according to an embodiment. The light source array 600 may be the light source array 140 shown in FIGS. 2-3 or the light source array 502 shown in FIGS. 6C-6D. The light source array 600 includes a plurality of LEDs 602 mounted on a thermally-conductive substrate 606 connected to a heat sink 604. The LEDs 602 are preferably high-intensity LEDs, each having an output of at least 3 watts, translating to a maximum irradiance of ˜30-150 mW/cm².

As shown, the LEDs 602 are arranged in a 6×3 grid. According to other embodiments, the light source array 600 may include additional rows or columns of LEDs forming a larger or smaller grid. Generally, the LEDs are arranged to form a region of uniform illumination (see FIGS. 2, 4C) that is at least 25 cm² in a horizontal plane, sufficient to illuminate all toenails simultaneously and uniformly.

Each LED 602 in the light source array 600 provided with a focusing lens to concentrate (i.e., intensify) light emitted by the LED 602 to a ˜45-degree beam angle to allow for a more uniform light distribution across the region of uniform illumination. The focusing lens 608 may be constructed of glass or plastic. According to some embodiments, the focusing lens 608 and may completely encapsulate the LED 602.

The plurality of LEDs 602 preferably comprises a first subset of LEDs emitting a first wavelength of light, and at least a second subset of LEDs emitting a second wavelength of light. The first and second subsets of LEDs may be arranged in alternating rows or columns in the light source array 600 or may be arranged in other arrangements. According to some embodiments, an LED emitter from the first subset and an LED emitter from the second subset may be combined into a single LED unit 602 provided with a common focusing lens 608.

Dual wavelength illumination may be beneficial for a combination of the therapeutic effect of each wavelength. For example, the first subset of LEDs may emit a wavelength of red light between 630 nm and 680 nm), to excite a fluorophore (e.g., Methylene Blue) in an topical formulation absorbed into the toenails to selectively mark/stain regions of onychomycosis infection; the second subset of LEDs may emit a wavelength of blue light between 400 nm and 420 nm for photodisinfection of onychomycosis and reduction of inflammation in the foot/toes/nailbed. The first and second subsets of LEDs may be configured to emit their respective wavelengths simultaneously, alternatingly (e.g., blue light, red light, blue light), or in a predetermined sequence (e.g., red light for 5 minutes, blue light for 10 minutes, red light and blue light for 10 minutes).

While the embodiments described herein illustrate use of the device in providing phototherapy to a user's foot, it will be understood by persons skilled in the art, that other body parts (e.g., fingers, hands) that can fit within the cavity, may be inserted for visualization and photodisinfection.

While the above description provides examples of one or more apparatus, methods, or systems, it will be appreciated that other apparatus, methods, or systems may be within the scope of the claims as interpreted by one of skill in the art.

1. A combined photodisinfection and visualization device for onychomycosis, comprising:
a housing having an open end for receiving a foot into a cavity of the housing;
a light source array embedded in at least a top surface of the cavity for directing a region of uniform illumination onto toenails, the light source array comprising a plurality of light emitting diodes (LEDs) configured to emit:
a first wavelength of light for excitation of a fluorophore absorbed into the toenails to selectively mark areas of onychomycosis infection; and
a second wavelength of light for reducing inflammation and for photodisinfection of the onycomycosis.

2. The device of claim 1, wherein the cavity encloses the lateral, medial and dorsal forefoot regions of the foot.

3. The device of claim 1, wherein the fluorophore is Methylene Blue.

4. The device of claim 1, wherein the plurality of LEDs comprise:
a first subset of LEDs for emitting the first wavelength of light; and
a second subset of LEDs for emitting the second wavelength of light.

5. The device of claim 1, wherein the first wavelength is between 630 nm and 670 nm and the second wavelength is between 400 nm and 420 nm.

6. The device of claim 1, wherein each LED comprises a focusing lens to concentrate light emitted by the LED to a ˜45-degree beam angle for forming the region of uniform illumination.

7. The device of claim 1, wherein the region of illumination covers a horizontal plane of at least 25 cm² to illuminate all the toenails simultaneously.

8. The device of claim 1, wherein the light source array is further embedded in opposing side walls of the cavity.

9. The device of claim 1, wherein the light source array is further embedded in a back wall of the cavity.

10. The device of claim 1, further comprising a cooling system having a heatsink connected to the light source array.

11. The device of claim 10, wherein the cooling system further comprises at least one fan configured to force air over the heatsink.

12. The device of claim 10, wherein the cooling system further comprises at least one fan configured to blow air into the cavity for cooling the foot.

13. The device of claim 1, further comprising: a detachable levelling mechanism disposed within the cavity, for aligning toes of the foot in a substantially horizontal plane, such that all the toenails are substantially equidistant from the light source array.

14. The device of claim 13, wherein the levelling mechanism is adjustable in height to vary a distance between the light source array and the toenails.

15. The device of claim 13, wherein the levelling mechanism comprises a ramped platform with an adjustable incline of at least 2 degrees.

16. The device of claim 1, further comprising extendable feet, wherein a distance between the light source array and the toes may be varied by extending or retracting the feet.

17. The device of claim 1, further comprising a detachable basin disposed within the cavity, for containing water or a solution for immersing the foot.

18. The device of claim 1, further comprising:
a camera disposed within the cavity, the camera configured to view the foot and/or the toes; and
an external display connected to the device, for displaying the camera view.

19. The device of claim 1, further comprising graduations or markings on the open end and/or on side walls of the cavity for positioning the toenails within the region of illumination.

20. The device of claim 1, further comprising a fluorescence sensor disposed within the cavity, for detecting and quantifying fluorescence emission of the fluorophore; and
an external display connected to the device, for displaying a fluorescence reading.
